(19) United States
(12) Patent Application Publication
(10) Pub. No.: US 2011/0117181 A1
(43) Pub. Date: May 19, 2011

(54) TREATING HEPATITIS C VIRUS INFECTION WITH OVER-EXPRESSION OF MICRORNA-196

(75) Inventors: Herbert L. Bonkovsky, Charlotte, NC (US); Weihong Hou, Weddington, NC (US)

(73) Assignee: The Charlotte-Mecklenburg Hospital Authority d/b/a Carolinas Medical Center

(21) Appl. No.: 12/575,884

(22) Filed: Oct. 8, 2009

(51) Int. Cl. A61K 9/127; A61K 31/7105; A61K 38/21; C12N 5/071; A61P 31/14

(52) U.S. Cl. 424/450; 514/44 A; 424/85.6; 435/370

(57) ABSTRACT

The present invention is directed to methods of treating cells infected with HCV and mammals suffering from HCV infection by transfecting the infected cells with miRNA-196 mimic. miRNA-196 mimic significantly down-regulates Bach1 protein and HCV gene expression, while also up-regulating HMOX1 gene expression. miRNA-196 binds with the 3′-UTR of Bach1 mRNA to reduce the expression of Bach1. As such, miRNA-196 can play an important role in the regulation of HCV replication and HMOX1/Bach1 expression in hepatocytes. The present invention also provides a formulation for the treatment of cells expressing HCV comprising a therapeutically effective amount of miRNA-196 such that Bach1 and HCV gene expression are down-regulated while HMOX1 expression is increased. The formulations are adapted to enable the transfection of miRNA-196 mimic into hepatocytes expressing HCV proteins.

### CROSS REFERENCE TO RELATED APPLICATIONS

This application is related to commonly owned copending Provisional Application Ser. No. 61/103,609, filed Oct. 8, 2008, incorporated herein by reference in its entirety, and claims the benefit of its earlier filing date under 35 U.S.C. 119(e).

### FEDERALLY SPONSORED RESEARCH OR DEVELOPMENT

This invention was made with United States Government support under R01-DK38825 awarded by NIH/NIDDK. The United States Government has certain rights in the invention.

### BACKGROUND OF THE INVENTION

1. Field of the Invention

The present invention relates generally to a method and formulation for the treatment of Hepatitis C infection. More particularly, the present invention relates to regulating Bach1 and/or HCV. Bach1 and the HCV NS5A protein can be down-regulated by miR-196, while up-regulating HMOX1.

2. Description of Related Art

Hepatitis C virus (HCV) is a small (50 nm in size), enveloped, positive sense single strand RNA virus in the family Flaviviridae. Although Hepatitis A virus, Hepatitis B virus, and Hepatitis C virus have similar names because they all cause liver inflammation, each is a distinct and different virus both genetically and clinically. HCV causes the blood-borne (i.e. spread by blood-to-blood contact) infectious disease known as Hepatitis C. The infection is often asymptomatic, but once established, chronic infection can cause inflammation of the liver (chronic hepatitis). This condition can progress to scarring of the liver (fibrosis), and advanced scarring (cirrhosis). In some cases, those with cirrhosis will go on to develop liver failure or other complications of cirrhosis, including liver cancer.

Acute hepatitis C refers to the first 6 months after infection with HCV. Around 60% to 70% of people infected develop no symptoms during the acute phase. In the minority of patients who experience acute phase symptoms, they are generally mild and nonspecific, and rarely lead to a specific diagnosis of hepatitis C. Symptoms of acute hepatitis C infection include decreased appetite, fatigue, abdominal pain, jaundice, itching, and flu-like symptoms. HCV is usually detectable in the blood within one to three weeks after infection, and antibodies to the virus are generally detectable within 3 to 12 weeks. Approximately 15-40% of persons infected with HCV clear the virus from their bodies during the acute phase as shown by normalization in liver function tests (LFTs) such as alanine transaminase (ALT) & aspartate transaminase (AST) normalization, as well as plasma HCV-RNA clearance (spontaneous viral clearance). The remaining 60-85% of patients infected with HCV develop chronic hepatitis C (CHC).

Chronic hepatitis C is defined as infection with HCV persisting for more than six months. The natural course of chronic hepatitis C varies considerably from one person to another. Virtually all people infected with HCV have evidence of inflammation on liver biopsy. However, the rate of progression of liver scarring (fibrosis) shows significant variability among individuals. Recent data suggests that among untreated patients, roughly one-third progress to liver cirrhosis in less than 20 years. Another third progress to cirrhosis within 30 years. Symptoms specifically suggestive of liver disease are typically absent until substantial scarring of the liver has occurred. However, hepatitis C is a systemic disease and patients may experience a wide spectrum of clinical manifestations ranging from an absence of symptoms to a more symptomatic illness prior to the development of advanced liver disease. Generalized signs and symptoms associated with chronic hepatitis C include fatigue, marked weight loss, flu-like symptoms, muscle pain, joint pain, intermittent low-grade fevers, itching, sleep disturbances, abdominal pain (especially in the right upper quadrant), appetite changes, nausea, diarrhea, dyspepsia, cognitive changes, depression, headaches, and mood swings.

Once chronic hepatitis C has progressed to cirrhosis, signs and symptoms may appear that are generally caused by either decreased liver function or increased pressure in the liver circulation, a condition known as portal hypertension. Possible signs and symptoms of liver cirrhosis include ascites (accumulation of fluid in the abdomen), bruising and bleeding tendency, bone pain, varices (enlarged veins, especially in the stomach and esophagus), fatty stools (steatorrhea), jaundice, and a syndrome of cognitive impairment known as hepatic encephalopathy.

Chronic hepatitis C, more than other forms of hepatitis, is diagnosed because of extrahepatic manifestations associated with the presence of HCV such as thyroiditis (inflammation of the thyroid) with hyperthyreosis or hypothyreosis, porphyria cutanea tarda, cryoglobulinemia (a form of small-vessel vasculitis) and glomerulonephritis (inflammation of the kidney), specifically membranoproliferative glomerulonephritis (MPGN). Hepatitis C is also associated with sicca syndrome, thrombocytopenia, lichen planus, diabetes mellitus and with B-cell lymphoproliferative disorders.

Hepatitis C virus infection is a world-wide health problem, for which a vaccine is currently not available. The current standard therapy for chronic hepatitis C(CHC) is a combination of pegylated interferon (IFN) and ribavirin, but only about 50% of patients respond to such treatment. Additionally, this treatment is expensive, prolonged, and accompanied by numerous unpleasant side-effects. An estimated 150-200 million people worldwide are infected with hepatitis C.

As such, there remains a need to discover and develop anti-viral therapies and procedures targeting HCV and for the treatment of all forms of Hepatitis C.

### BRIEF SUMMARY OF THE INVENTION

The present invention satisfies at least some of the aforementioned needs by providing a method of treating cells or a mammal suffering from HCV infection by reducing the expression of Bach1 protein levels in human hepatoma cells expressing hepatitis C viral proteins. A reduction in the expression of Bach1 protein levels can be achieved by transfecting the cells with miRNA-196 mimic so that the miRNA-196 binds with the 3′-UTR of Bach1 mRNA to reduce the expression of Bach1. The miRNA merely needs to include the matching “seed region” to effectively bind with the 3′-UTR of Bach1 mRNA. Preferably, the miRNA is up-regulated or over-expressed to increase the reduction in Bach1 expression levels. The level of miRNA-196 is up-regulated by a synthesized miRNA mimic, which enters the miRNA pathway and acts as a mature miRNA-196.

A mammal suffering from HCV infection can also be treated by up-regulating HMOX1 gene expression in cells expressing HCV non-structural proteins. In various embodiments, the up-regulation of HMOX1 gene expression is accompanied by the down-regulation of Bach1 gene expression in the cells. As such, the miRNA-196 indirectly up-regulates HMOX1 by binding with Bach1, which negatively regulates HMOX1. The regulation of each can be achieved by transfecting the cells with miRNA-196 mimic. Additionally, cells, such as hepatocytes, infected with HCV can be treated by reducing the expression of HCV in the infected cells by transfecting the cells with miRNA-196 mimic.

In another aspect, the invention provides a pharmaceutical formulation adapted for administering miRNA-196 mimic to a mammal so that cells infected with HCV can be transfected with miRNA-mimic. The administration of these formulations translationally represses the expression of Bach1, up-regulates HMOX1, and regulates HCV replication in hepatocytes.

Importantly, the present invention demonstrates the functional miRNA-196 binding sites in the 3′-UTR of Bach1, which lead to down regulation of Bach1 gene expression, up regulation of HMOX1 gene expression, and down regulation of HCV gene expression. The use of miRNA-196 provides new additional therapies for treating cells infected with HCV and mammals having Hepatitis C (e.g. chronic HCV infection) and, perhaps, for other diseases characterized by increased oxidative stress.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1A shows a schematic of a first seed region match between miR-196 and the first putative Bach1 3′-UTR site targeted;

FIG. 1B shows a schematic of second seed region match between miR-196 and the putative Bach1 3′-UTR site targeted;

FIG. 2A illustrates the down-regulated Bach1 protein levels associated with transfection with miRNA-196 mimic;

FIG. 2B illustrates up-regulated Bach1 protein levels associated with transfection with miRNA-196 inhibitor;

FIG. 2C shows that Bach1 mRNA levels were not altered by miRNA-196 mimic transfection;

FIG. 3A illustrates the up-regulation of HMOX1 mRNA levels associated with miRNA-196 mimic;

FIG. 3B shows that transfection with miRNA-196 mimic did not alter Cullin 3 mRNA levels;

FIG. 4A illustrates the down-regulation of HCV NS5A protein levels associated with transfection with miRNA-196 mimic;

FIG. 4B illustrates the up-regulation of HCV NS5A protein levels associated with transfection with miRNA-196 inhibitor;

FIG. 4C shows the down-regulation of HCV NS5A and core mRNA levels by transfection with miRNA-196 mimic in the Con 1 (subtype 1b) full length replicon cells;

FIG. 5A shows the two Bach1 3′-UTR seed match sites for miRNA-196;

FIG. 5B illustrates a schematic representation of pGL3-Bach1, the firefly luciferase (f-luc) reporter construct utilized in co-transfectin cells with pGL3-Bach1, pRL-TK (renilla) and with miR-196 mimic or inhibitor by Lipofectamine 2000.

FIG. 5C shows that miRNA-196 mimic inhibited the f-luc activities of pGL3-Bach1 reporter;

FIG. 5D shows that miRNA-196 inhibitor slightly increased the f-luc activity of pGL3-Bach1 reporter;

FIG. 6A illustrates the replacement of four nucleotides in the two seed match sites of Bach1 3′-UTR;

FIG. 6B illustrates that miRNA-196 mimic decreased the f-luc activity of pGL3-Bach1-WT but not pGL-Bach1-Mut reporter in cells co-transfected with mutant pGL3-Bach1 or pGL3-Bach1, with pRL-TK (renilla), and with miR-196 mimic;

FIG. 6C illustrates that miR-155 mimic decreased the f-luc activities of both pGL3-Bach1-WT and pGL-Bach1-Mut reporter in cells co-transfected with mutant pGL3-Bach1 or pGL3-Bach1, with pRL-TK (renilla), and with miR-155 mimic;

FIG. 7A illustrates the four nucleotide mutations that were introduced to the seed match sites of miRNA-196;

FIG. 7B shows the Luciferase activity of cells co-transfected with pGL3-Bach1, pRL-TK and with increasing concentrations of mimic negative control, miR-196 mimic or mutant miR-196;

FIG. 8A illustrates the restoration of the seed match between mutant miRNA-196 and mutant Bach1 3′-UTR;

FIG. 8B shows the measured luciferase activities of cells co-transfected with mutant reporter (pGL3-Bach1-Mut), pRL-TK, and with mutant miRNA-196 or wild type miRNA-196 mimic for 48 h;

FIG. 9A shows down-regulation of HCV J6/JFH1 RNA levels by miRNA-196 mimic in Huh-7.5 cells transfected with J6/JFH1 RNA;

FIG. 9B shows in Huh-7.5 cells infected with J6/JFH1 hepatitis C virus secreted into the culture supernatant; and

FIG. 9C shows down-regulation of HCV J6/JFH1 protein levels by miRNA-196 mimic in Huh-7.5 cells infected with J6/JFH1 hepatitis C virus secreted into the culture supernatant.

### DETAILED DESCRIPTION OF THE INVENTION

The present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements.

It has been accepted that Hepatitis C virus (HCV) directly induces oxidative stress in hepatocytes. Equally accepted, heme oxygenase 1 (HMOX1) is a key cytoprotective enzyme that has anti-oxidant and anti-inflammatory activities. As such, the existence or use of HMOX1 can be viewed as a means for negating or mitigating the oxidative stress induced by HCV. However, Bach1, a basic leucine zipper (bZip) mammalian transcriptional repressor, negatively regulates HMOX1. As such, reduction of Bach1 levels, alone or in conjunction with increased HMOX1 levels, can potentially lead to the prevention or amelioration of Hepatitis C.

Bach1 is a gene that encodes a transcription factor that belongs to the cap‘n’collar type of basic region leucine zipper factor family (CNC-bZip). The encoded protein contains broad complex, tramtrack, bric-a-brac/poxvirus and zinc finger (BTB/POZ) domains, which is atypical of CNC-bZip family members. These BTB/POZ domains facilitate protein-protein interactions and formation of homo- and/or hetero-oligomers. When this encoded protein forms a heterodimer with MafK, it functions as a repressor of Maf recognition element (MARE) and transcription is repressed. More pointedly, Bach1 is a mammalian transcriptional repressor of HMOX1 that negatively regulates HMOX1 gene expression. Bach1 forms antagonizing heterodimers with the Maf-related oncogene family. These heterodimers bind to Maf recognition elements (MARES) and suppress expression of genes (e.g., HMOX1 and NQO1) that respond to Maf-containing heterodimers and other positive transcriptional factors.

miRNAs are small non-coding RNAs (˜22 nt) that are generally considered to be important regulators of gene expression. Prior to the present invention, whether and how microRNAs regulate Bach1 or HCV was largely unknown. The present invention recognizes, for the first time, that miRNA-196 directly acts on the 3′-UTR of Bach1 mRNAs and translationally represses the expression of this protein, and up-regulates HMOX1. Additionally, miR-196 also inhibits HCV NS5A protein expression. Thus, miRNA-196 plays an important, perhaps even critical, role in the regulation of HCV replication and HMOX1/Bach1 expression in hepatocytes. Accordingly, cells infected with HCV or mammals suffering from Hepatitis can be treated by the administration of miRNA-196 mimic so that the infected cells are transfected with miRNA-196 mimic. Preferably, over-expression of miRNA-196 and tranfection thereof into infected cells can beneficially provide an approach for preventing or ameliorating hepatitis C infection. In one embodiment, the level of miRNA-196 is up-regulated by a synthesized miRNA mimic, which enters the miRNA pathway and acts as a mature miRNA-196.

As referenced above, miRNAs are small non-coding RNAs (˜22 nt) that are generally considered to be important regulators of gene expression. More specifically, miRNAs are single-stranded RNA molecules of about 21-23 nucleotides in length, which regulate gene expression primarily through translational repression. miRNAs are encoded by genes that are transcribed from DNA but not translated into protein (non-coding RNA); instead they are processed from primary transcripts known as pri-miRNA to short stem-loop structures called pre-miRNA and finally to functional miRNA. Mature miRNA molecules are partially complementary to one or more messenger RNA (mRNA) molecules, and their main function is to downregulate gene expression. The function of miRNAs appears to be in gene regulation. For that purpose, a miRNA is complementary to a part of one or more messenger RNAs (mRNAs). Animal miRNAs are usually complementary to a site in the 3′ UTR. The annealing of the miRNA to the mRNA then inhibits protein translation, but sometimes facilitates cleavage of the mRNA. In such cases, the formation of the double-stranded RNA through the binding of the miRNA triggers the degradation of the mRNA transcript through a process similar to RNA interference (RNAi). However, in other cases it is believed that the miRNA complex blocks the protein translation machinery or otherwise prevents protein translation without causing the mRNA to be degraded. Currently, the exact mechanism by which target genes are down-regulated remains unclear.

For purpose of the present application, MicroRNA mimics (e.g. miRNA-196 mimic) are double-stranded RNA oligonucleotides chemically modified with ON-TARGET® to increase their stability and to improve their activities. The microRNA mimics mimic endogenous precursor miRNAs to enter the miRNA pathway and act as mature miRNA species.

Sequences complementarity in the 6-8 base pair ‘seed regions’ at the end of the miRNA-mRNA heteroduplex appear to determine the specificity of miRNA-target RNA interactions. miR-196 was first recognized to have extensive and evolutionarily conserved complementarity to homeobox (HOX) clusters, groups of related transcription factor genes crucial for numerous of developmental programs in animals, and to regulate HOX gene expression.

The present inventors propose that miRNA-196 targets HCV genome as well as up-regulation of the HMOX1 gene by targeting the 3′-UTR of Bach1 mRNA. Accordingly, cells infected with HCV can be treated by tranfecting the infected cells with miRNA-196 mimic. By transfecting the HCV infected cells, Bach1 expression levels can be reduced while increasing HMOX1 levels. Beneficially, the down-regulation of Bach1, which negatively regulates HMOX1, aids in increasing the expression of HMOX1. As noted above, HMOX1 provides anti-oxidative effects to mitigate the oxidative stress induced by HCV. Additionally, miRNA-196 up-regulation can be induced with IFN β treatment. This induction of miRNA-196 can be seen in the human hepatoma cell line Huh-7 and in primary murine hepatocytes.

The numbers of miRNAs continues to grow, and additional mRNAs and candidate genes regulated by them continue to be identified. With respect to the liver, miRNA-122 was identified as the most abundant miRNA expressed in hepatocytes and shown to have major effects on several enzymes of cholesterol metabolism. miRNA-122 was also shown to be required for HCV expression. Generally, the effects of miRNA-122 depend upon the context and location of its cognate seed sequence binding sites with the sites in the 5′ region mostly associated with up-regulation of expression, whereas those in the 3′ untranslated region mostly associated with repression of expression. Embodiments of the present invention comprise utilizing miRNA-196 mimic as a down-regulator of HCV NS5A protein expression. This protein is essential for the full and normal expression of HCV. As such, embodiments of the present invention comprise methods of treating infected cells or mammals suffering from Hepatitis C with a therapeutically effective amount of miRNA-196 mimic. A therapeutically effective amount can be provided by known administrative routes. For instance, therapeutic agents comprising miRNA-196 mimic can be applied by topical, enteral or parenteral administration routes according to various embodiments of the present invention.

It is now generally accepted that HCV infection produces an increase in oxidative stress in infected hepatocytes and probably in other infected cells as well. One important mediator of such increased oxidative stress is the HCV core protein. Equally accepted, HMOX1 helps to protect numerous cells and tissues against the potentially damaging effects of excess oxidative stress. As referenced above, HMOX1 is a key cytoprotective enzyme that has anti-oxidant and anti-inflammatory activities that may negate or mitigate the oxidative stress induced by HCV. More specifically, HMOX1 is a key cytoprotective enzyme, catalyzing heme degradation and generating ferrous iron, carbon monoxide and biliverdin, which have anti-oxidant and anti-inflammatory activities in vivo. These beneficial properties of HMOX1 are based upon the ability of HMOX1 to decrease “free” or loosely-bound heme, which can act as a potent prooxidant, and to increase production of carbon monoxide, biliverdin, and bilirubin, which have potent antioxidant and anti-inflammatory and antifibrogenic effects.

Regulation of expression of the HMOX1 gene is complex. However, the present inventors have shown that among the important sites for regulation of HMOX1 are a series of expanded AP-1 sites (also called antioxidant responsive elements), Maf protein responsive elements [MARE], and metalloporphyrin-responsive elements [MPRE] in the 5′ untranslated region of HMOX genes across many species. Bach1 is a member of the cap n' collar family zinc, leucine zipper proteins. It plays a key role in tonic repression of expression of the HMOX1 gene. It does so by forming heterodimers with small Maf proteins and blocking transcriptional activation of the gene. Bach1 contains several consensus binding sites (all containing CP motifs) which, when they bind heme, lead to a change in conformation of the protein with marked reduction in affinity for Maf proteins and subsequent depression and increase in activity of HMOX1 gene expression. One of the major stimulatory Maf proteins is Nrf2. Up regulation of Nrf2 is associated with increased expression of the HMOX1 gene.

In view of the above, HMOX1 activity might be increased in HCV infection. Nevertheless, clinical studies have identified a decrease in expression of HMOX1 in the setting of chronic hepatitis C. As such, patients with genetic or other factors that lead to lower levels of HMOX1 gene expression may be at increased risk for development of chronic hepatitis C infection after acute HCV exposure and/or with greater risks of development of more rapidly progressive liver disease due to HCV infection.

Lower levels of HMOX1 gene expression can be correlated to chronic hepatitis C infection and Bach1 negatively regulates HMOX1. Accordingly, treatment of cells infected with HCV or mammals suffering from Hepatitis C in a manner to reduce the level of Bach1 in the infected cells can beneficially mitigate the impact of HCV. As such, one embodiment of the present invention comprises a method for treating a mammal suffering from chronic hepatitis C infection by transfecting the cells with miRNA-196 mimic so that the miRNA-196 binds with the 3′-UTR of Bach1 mRNA to reduce the expression of Bach1 and up-regulate HMOX1. Preferably, the miRNA-196 is over expressed to provide more miRNA-196 for binding to Bach1 and up-regulating HMOX1.

In one embodiment according to the present invention, a mammal suffering from HCV infection (e.g. chronic hepatitis C) is treated with miRNA-196. In particular, the infected cells are transfected with miRNA-196 mimic, which down-regulates the expression of Bach1. In one particular embodiment, Bach1 protein expression levels in infected cells expressing HCV non-structural protein can be reduced by transfecting the cells with miRNA-196 mimic so that the miRNA-196 binds with the 3′-UTR of Bach1 mRNA to reduce the expression of Bach1. In one preferred method, the miRNA-196 which binds with the 3′-UTR of Bach1 mRNA is over-expressed. In one particular embodiment, miRNA-196 is up-regulated by also administering interferon beta. In another embodiment, miRNA-196 is also up-regulated by the administration of miRNA-196 mimic alone or in combination with interferon beta.

The treatment of infected cells individually, or mammals carrying such infected cells, can provide a significant reduction in the expression of Bach1 protein levels upon the administration of a therapeutically effective amount of miRNA-196 mimic. By “therapeutically effective amount” it is meant an amount of miRNA-196 mimic to an infected cell or mammal having Hepatitis effective to treat and/or prevent one or more targeted disorders. By way of general guidance, a daily therapeutic amount of the miRNA-196 mimic for the treatment of HCV infection can generally range from 0.5 to 5 μmol/kg of body weight, or from 1.0 to 4 μmol/kg of body weight, or from 2 to 3 μmol/kg of body weight. In an alternative embodiment, the therapeutic amount of miRNA-196 mimic can range from 0.1 to 1 μmol/kg of body weight, or from 0.25 to 1 μmol/kg of body weight, or form 0.25 to 0.75 μmol/kg of body weight.

In various embodiments, the expression of Bach1 protein levels can be reduced by about 10% to about 75%, or by about 25% to about 65%. In other embodiments, the expression of Bach1 protein levels 24 hours after transfection comprises a reduction of about 25% to about 75%, or about 50% to about 60%. According to yet another embodiment, the expression of Bach1 protein levels 48 hours after transfection provides a reduction of about 40% to about 80%, or about 60% to about 70%.

As discussed above, increasing the level of HMOX1 gene expression can be beneficial in negating or mitigaiting the oxidative stress induced by HCV. When transfected with miRNA-196, HMOX1 gene expression in cells expressing HCV non-structural proteins can be beneficially increased. Accordingly, one embodiment of the present invention comprises a method of treating cells infected with HCV or a mammal suffering from HCV infection by up-regulating HMOX1 gene expression in cells expressing HCV non-structural proteins. HMOX1 gene expression is up-regulated by transfecting the infected cells with a therapeutically effective amount of miRNA-196 mimic. In one embodiment the up-regulating of HMOX1 is accompanied with the down-regulating of Bach1 gene expression in the cells. In one preferred embodiment, the method includes up-regulating or over-expressing miRNA-196 to treat hepatocytes. In various embodiments, the expression of HMOX1 is increased by about 2 to about 3 fold over the level of HMOX1 expression in cells that have not been transfected with miRNA-196 mimic.

In addition to repressing Bach1 expression, HCV non-structural protein 5a (HCV NS5A) expression in human hepatocytes expressing HCV non-structural proteins is also repressed. As such, embodiments of the present invention comprise treating HCV infected cells with miRNA mimic. The infected cells can be tranfected with miRNA-196 mimic to down-regulate the expression of HCV NS5A. Preferably, the miRNA-196 is over-expressed.

Certain embodiments of the present invention comprises treating cells infected with HCV or a mammal suffering from HCV infection by reducing the expression of HCV NS5A in cells expressing HCV non-structural proteins by transfecting the cells with miRNA-196 mimic. That is, in certain embodiments a mammal suffering from HCV infection is treated by reducing the expression of HCV RNA and/or protein expression in HCV replicon cells and HCV infected cells by transfecting the cells with miRNA-mimic. In one such embodiment, the expression of HCV NS5A protein levels is reduced by 10 to 60 percent, or by 20 to 50 percent. In another embodiment, the expression of HCV NS5A protein levels 24 hours after transfection comprise a reduction of about 45 to about 55 percent, while the expression of HCV NS5A protein levels 48 hours after transfection comprise a reduction of about 35 to about 45 percent.

The term “transfection” as used throughout the present application describes the introduction of foreign material into eukaryotic cells using a virus vector or other means of transfer. Transfection of animal cells typically involves opening transientpores or ‘holes’ in the cell plasma membrane, to allow the uptake of material. Genetic material (such as supercoiled plasmid DNA or siRNA constructs), or even proteins such as antibodies, may be transfected. In addition to electroporation, transfection can be carried out by mixing a cationic lipid with the material to produce liposomes, which fuse with the cell plasma membrane and deposit their cargo inside.

One possible method of transfection can utilize calcium phosphate. A HEPES-buffered saline solution (HeBS) containing phosphate ions is combined with a calcium chloride solution containing the material to be transfected. When the two are combined, a fine precipitate of the positively charged calcium and the negatively charged phosphate will form, binding the material to be transfected on its surface. The suspension of the precipitate is then added to the cells to be transfected. Although the process is not completely understood, the cells take up some of the precipitate, and with it, the material to be transfected.

Other methods use highly branched organic compounds (e.g. dendrimers) to bind the genetic material (e.g. DNA, RNA, or miRNA) and get it into the cell. Another method is the inclusion of the genetic material to be transfected in liposomes, i.e. small, membrane-bounded bodies that are in some ways similar to the structure of a cell and can actually fuse with the cell membrane, releasing the genetic material into the cell. For eukaryotic cells, lipid-cation based transfection is more typically used, because the cells are more sensitive.

Another method is the use of cationic polymers such as DEAE-dextran or polyethylenimine. The negatively charged genetic material binds to the polycation and the complex is taken up by the cell via endocytosis.

A direct approach to transfection is the gene gun, where the genetic material is coupled to a nanoparticle of an inert solid (commonly gold) which is then “shot” directly into the target cell's nucleus. The genetic material can also be introduced into cells using viruses as a carrier. In such cases, the technique is called viral transduction, and, the cells are said to be transduced. Other methods of transfection include nucleofection, electroporation, heat shock, magnetofection and proprietary transfection reagents such as Lipofectamine, Dojindo Hilymax, Fugene, jetPEI, Effectene or DreamFect.

U.S. Pat. No. 5,942,634, incorporated herein by reference, describes the use of cationic amphiphiles to facilitate transport of biologically active (therapeutic) molecules into cells. U.S. Pat. No. 5,942,634 also teaches how to make therapeutic compositions incorporating a therapeutic molecule by contacting a dispersion of one or more cationic amphiphiles with the therapeutic molecules. The therapeutic molecules that can be delivered into cells include DNA, RNA, and polypeptides. Such compositions can be used to provide gene therapy, and delivery of antisense polynucleotides or biologically active polypeptides to cells.

In yet another aspect, the present invention provides a pharmaceutical formulation for the treatment of cells or mammals having hepatitis C, preferably chronic hepatitis C. Formulations according to the present invention should comprise a therapeutically effective amount of miRNA-196 mimic so the Bach1 and/or HCV NS5A are down-regulated while HMOX1 expression is increased. Embodiments according to the present invention are adapted to enable the transfection of miRNA-196 mimic into hepatocytes expressing HCV. The formulations of the present invention can include or utilize, but are not limited to, any of the aforementioned means for transfecting genetic material into a target cell.

According to various embodiments, formulations of the present invention can be provided in a form for enteral administration. For instance, a formulation according to embodiments of the present invention can be provided in the form of a tablet, capsule or liquid preparation for oral administration. Most preferably, however, the formulation is provided as a liquid preparation for intravenous injection.

In other embodiments, the formulation can be provided in a form for injection or infusion. For example, various formulations according to the present invention can be administered intravenous (into a vein), intraarterial (into an artery), intramuscular (into a muscle), subcutaneous (under the skin), intradermal, (into the skin itself), intrathecal (into the spinal canal), or intraperitoneal, (infusion or injection into the peritoneum)

The treatment of infected cells individually, or mammals carrying such infected cells, with the formulations according to embodiments of the present invention can provide a significant reduction in the expression of Bach1 and HCV NS5A protein levels and an increase in HMOX1 levels upon the administration of a therapeutically effective amount of miRNA-196 mimic. By “therapeutically effective amount” it is meant an amount of miRNA-196 mimic to an infected cell or mammal having Hepatitis effective to treat and/or prevent one or more targeted disorders. By way of general guidance, a daily therapeutic amount of the miRNA-196 mimic for the treatment of HCV infection can generally range from 0.5 to 5 μmol/kg of body weight, or from 1.0 to 4 μmol/kg of body weight, or from 2 to 3 μmol/kg of body weight. In an alternative embodiment, the therapeutic amount of miRNA-196 mimic can range from 0.1 to 1 μmol/kg of body weight, or from 0.25 to 1 μmol/kg of body weight, or form 0.25 to 0.75 μmol/kg of body weight.

### Working Examples

### I. Materials and Experiments

### A. Reagents and Antibodies

BCA protein assay reagents were obtained from Pierce Biotechnology (Rockford, Ill.). Dulbecco's modified Eagle's medium (DMEM) and fetal bovine serum (FBS) were from HyClone (Logan, Utah). The Dual-Glo® Luciferase Assay System was from Promega (Madison, Wis.). TRIzol was purchased from Invitrogen (Carlsbad, Calif.) and geneticin (G-418) was from Gibco (Grand Island, N.Y.). Primers were synthesized by Integrated DNA Technologies (Coralville, Iowa). 4-15% gradient SDS-PAGE gels and ImmunBlot PVDF membranes were purchased from Bio-Rad (Hercules, Calif.). Mouse anti-HCV NS5A and mouse anti-HCV NS3 monoclonal antibodies were purchased from Virogen (Watertown, Mass.). Goat anti-human Bach1 and GAPDH polyclonal antibodies were from Santa Cruz Biotechnology (Santa Cruz, Calif.). ECL-Plus was from Amersham (Piscataway, N.J.).

### B. Cell Culture

9-13 cells were provided by University of Heidelberg, Heidelberg, Germany. The 9-13 cell line, a human hepatoma Huh-7 cell population, harbors a replicating HCV non-structural region and stably expresses HCV non-structural protein NS3 to NS5B. The cells were maintained in DMEM supplemented with 10% (v/v) FBS, 100 u/mL penicillin, 100 μg/mL streptomycin, and 500 μg/mL G-418. The Con 1 (subtype 1b) full length replicon Huh-7.5 cells (Con1 cells) was from the Rockefeller University (New York, N.Y.). The Con1 cell line is a Huh-7.5 cell population containing the full-length HCV genotype 1b replicon with the highly adaptive serine to isoleucine substitution at amino acid 2204 of the polypeptide. The Con1 cells were maintained in DMFM supplemented with 10% (v/v) FBS and 0.1 mM nonessential amino acids, 100 units/mL penicillin, 100 μg/mL streptomycin, and selection antibiotic 750 μg/mL G418. The cells were maintained in a humidified atmosphere of 95% room air and 5% CO₂ at 37° C.

C. microRNAs and Constructs

The miRIDIAN microRNA mimics for has-miRNA-196, has-miRNA-16, customized mutant has-miRNA-196, and miRNA mimic negative control (MMNC) were obtained from Dharmacon (Lafayette, Colo.). MicroRNA mimics are double-stranded RNA oligonucleotides chemically modified with ON-TARGETS to increase their stability and to improve their activities. The microRNA mimics mimic endogenous precursor miRNAs to enter the miRNA pathway and act as mature miRNA species. Additionally, the following miRNA inhibitors, has-miR-196 and miRNA inhibitor negative control (MINC), were also from Dharmacon. miRIDIAN microRNA hairpin inhibitors are the newest generation synthetic oligonucleotide inhibitors designed to suppress the native miRNAs activity. pRL-TK vector was from Promega. The pRL-TK reporter vector contains a cDNA (Rluc) encoding Renilla luciferase as an internal control reporter. pGL3-Bach1 luciferase reporter construct, containing an 1837 bp fragment of Bach1 3′-UTR, was provided by University of Florida, Gainesville, Fla. Mutant pGL3-Bach1 was generated by GENEWIZ, Inc. (South Plainfield, N.J.). Constructs were confirmed by restriction enzyme digestion and sequencing.

### D. Transfection and Luciferase Activity Assays

Transfections were carried out using Lipofectamine 2000 (Invitrogen) according to manufacturer's protocols. Briefly, 9-13 cells were co-transfected with pGL3-Bach1 or mutant pGL3-Bach, with pRL-TK, and with tested miRNAs. Forty-eight hours after transfection, cells were harvested and lysed. The luciferase reporter activities were measured using the Dual-Glo® Luciferase Assay System. Firefly luciferase activity was normalized to Renilla luciferase activity and total protein determined using the BCA protein assay kit. For ease of comparison, values for cells with pGL3-Bach1 and pRL-TK transfection were set equal to 1.

### E. In Vitro Transcription, Transfection and Infection

The HCV infectious clone pJ6/JFH1, the full-length chimeric genome with the core-NS2 regions from the infectious J6 (genotype 2a) and NS3-NS5B regions from the infectious JFH1 (genotype 2a), was generously provided by C. M. Rice (the Rockefeller University, New York, N.Y.). The production of J6/JFH1-based cell culture-generated HCV (HCVcc) has been reported previously. In brief, the pJ6/JFH1 plasmid was linearized with XbaI, and purified by ethanol precipitation, digestion with proteinase K, and phenol-chloroform extraction. The linearized plasmid was used as a template for in vitro transcription using the MEGAscript T7 kit (Ambion, Austin, Tex.). For HCV RNA transfection, Huh-7.5 cells were plated in 24-well plates one day prior to transfection and transfected at 70-80% confluence. Cells were transfected at an RNA/lipofectamine ratio of 1:2 by using 2 μg/well of HCV RNA and 4 uL/well Lipofectamine 2000 for 48 h. To infect naïve Huh-7.5 cells, cell culture supernatants from the cells transfected with HCV RNA for 48 h were collected and filtered through a 0.20 μm filter, and added to cultures of naïve Huh-7.5 cells.

### F. Western Blots

Western blots were performed as described previously. In brief, total proteins (30-50 μg) were separated on SDS-PAGE gels. After electrophoretic transfer onto ImmunBlot PVDF membranes, membranes were blocked for 1 hour in PBS containing 5% nonfat dry milk and 0.1% Tween-20, and then incubated overnight with primary antibody at 4° C. The dilutions of the primary antibodies were as follows: 1:1000 for anti-Bach1 antibody, and 1:2000 for anti-HCV NS5A and anti-GAPDH antibodies. The membranes were then incubated for 1 hour with horseradish peroxidase-conjugated secondary antibodies (dilution 1:10,000). Finally, the bound antibodies were visualized with the ECL-Plus chemiluminescence system according to the manufacturer's protocol. A Kodak 1DV3.6 computer-based imaging system (Rochester, N.Y.) was used to measure the relative optical density of each specific band obtained after Western blotting.

### G. Quantitative RT-PCR

Total RNA from tested cells was extracted and cDNA was synthesized as described previously. Primers used were as follows: Bach-1-specific sense primer 5′-GGACACTCCTTGCCAAATGCAG-3′ (22 bp), anti-sense primer 5′-TGACCTGGTTCTGGGCTCTCAC-3′ (22 bp); HMOX1-specific sense primer 5′-CGGGCCAGCAACAAAGTG-3′ (18 bp), anti-sense primer 5′-AGTGTAAGGACCCATCGGAGAA-3′ (22 bp); Cu13-specific sense primer 5′-GTGCTCACGACAGGATA-3′ (17 bp), anti-sense primer 5′-GTTTGGCTAAGTAGAACCTTC-3′ 21 bp); GAPDH-specific sense primer 5′-TTGTTGCCATCAATGACCC-3′ (19 bp), anti-sense primer 5′-CTTCCCGTTCTCAGCCTTG-3′ (19 bp). Real time quantitative RT-PCR was performed using a CFX96™ Real-Time PCR Detection System (Bio-Rad) and iQ® SYBR Green Supermix Real-Time PCR kit (Bio-Rad). Samples were included without template and without reverse transcriptase as negative controls, which, as expected, produced negligible signals (Ct values>35). Fold-change values were calculated by comparative Ct analysis after normalizing for the quantity of GAPDH in the same samples. Initial experiments showed that the several treatments and manipulation of cells did not affect the expression of GAPDH gene.

### H. Statistical Analysis

Initial analysis showed that results were normally distributed. Therefore, parametric statistical procedures were used. The Student's t-test (for comparison of two conditions) or analysis-of-variance (for comparison among more than two) was used to analyze the differences between samples. Values of P<0.05 were considered statistically significant. Experiments were repeated at least three times with similar results. All experiments included at least triplicate samples for each treatment group. Representative results from single experiments are presented. Statistical analyses were performed with JMP 4.0.4 software from SAS Institute (Cary, N.C.).

### II. Results

A. Analysis in Silico Predicts Two Seed Region Matches of miR-196 and the 3′-UTR of Bach1 mRNA

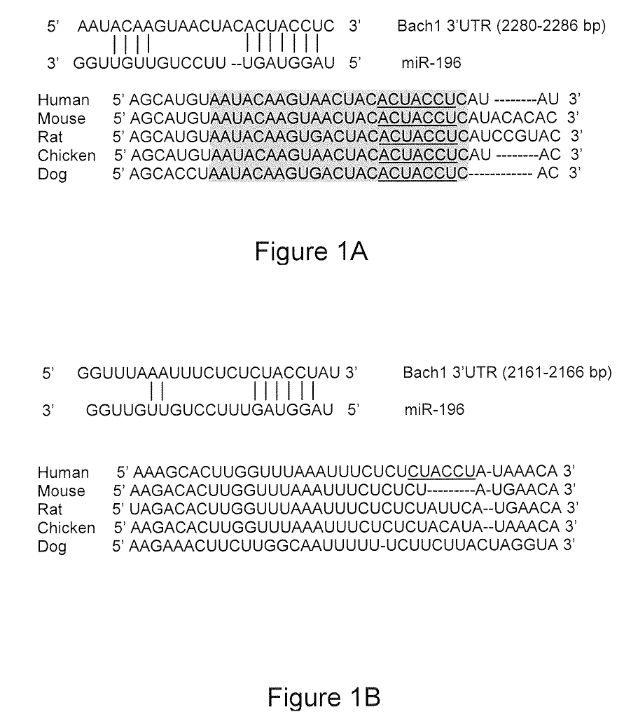

Bioinformatic approaches were utilized to identify potential miRNA targets. An online search of the TargetScan 4.0 data base demonstrated that at least two putative miRNA-196 seed match sites were harbored in the 3′-UTR of Bach1 mRNA. As shown in FIGS. 1A & B, one of the predicted binding site (2280-2286 nt) was highly conserved in human, mouse, rat, chicken and dog, whereas the other putative sites (2161-2166 nt) was poorly conserved across species. No predicted miRNa-196 binding sites were found in the Nrf2 and HMOX1 gene, and no putative miRNA-196 binding sites were found in the coding region of Bach1 gene (data not shown).

B. miRNA-196 Down-Regulates the Transcriptional Repressor Bach1 and Up-Regulates HMOX1 in 9-13 Cells Expressing HCV Non-Structural Proteins

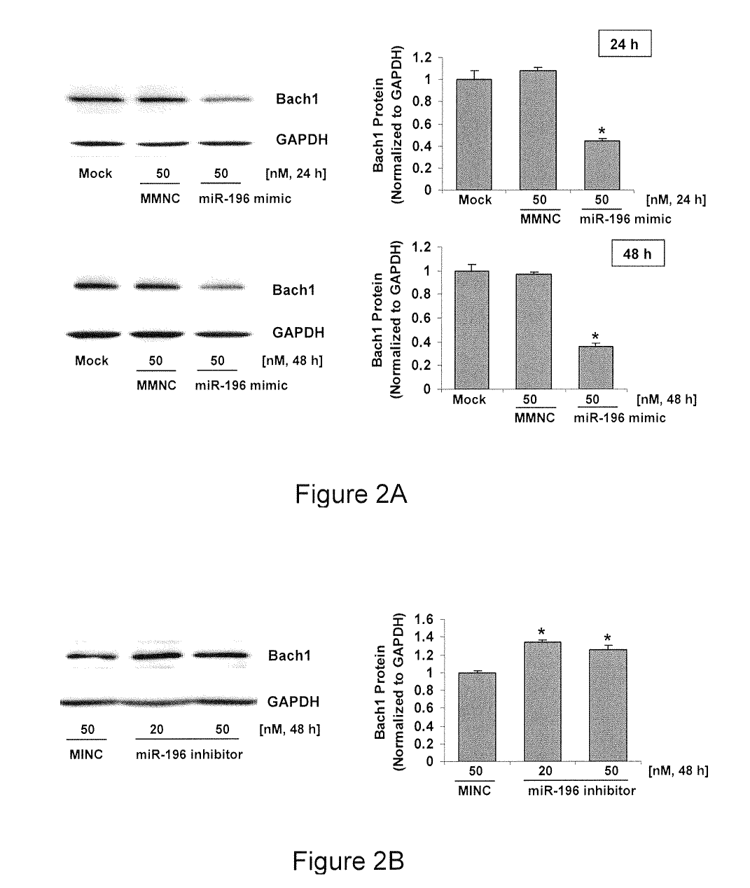

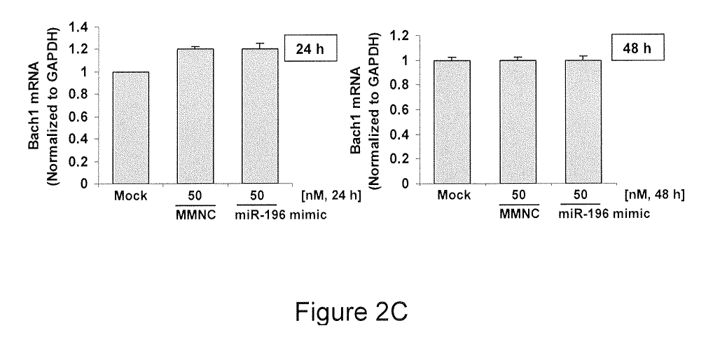

To experimentally verify that the putative miR-196 binding sites are functional, the 9-13 cells were transfected with miRNA-196 specific mimic or inhibitor. Bach1 protein and mRNA levels were assessed by Western blots and qRT-PCR, respectively. 9-13 cells transfected with miRNA-196 mimic showed a significant reduction in the expression of Bach1 protein levels by about 55% after 24 h transfection and about 64% after 48 h transfection compared with miRNA mimic negative control (MMNC). No effects on Bach1 protein levels were detectable in cells transfected with miRNA mimic negative control compared with mock transfection (FIG. 2A). Down-regulation of miRNA-196 by miRNA-196 specific inhibitor significantly increased Bach1 protein levels in contrast to miRNA inhibitor negative control (FIG. 2B). However, no significant effect of miRNA-196 on Bach1 mRNA levels was observed in 9-13 cells. The levels of Bach1 mRNA were not altered with miRNA-196 mimic transfection (FIG. 2C). These results demonstrated that the regulation of miRNA-196 on Bach1 may occur at a translational level in human hepatoma 9-13 cells.

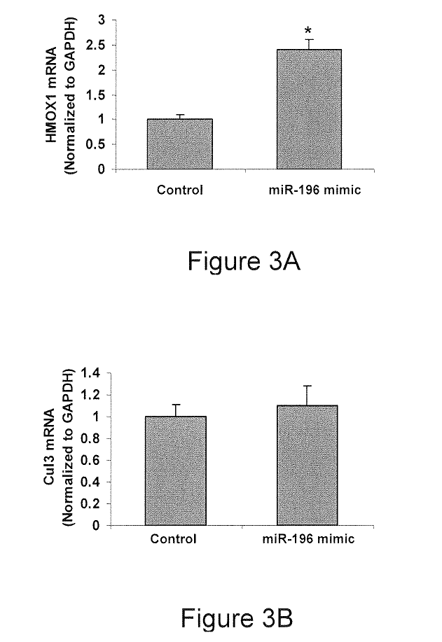

Since Bach1 is a well-established transcriptional repressor of the HMOX1 gene, the present inventors next determined whether down-regulation of Bach1 protein by miRNA-196 could increase HMOX1 gene expression. Thus, 9-13 cells were transfected with miRNA-196 mimic or miRNA mimic negative control for 48 h. After 48 h, the levels of HMOX 1 and Cullin 3 (Cu1 3, non-specific gene control) mRNA were quantified by qRT-PCR. miRNA-196 mimic significantly up-regulated HMOX1 mRNA levels by 2.4 fold compared with the same amount of miRNA mimic negative control as shown in FIG. 3A. Cu1 3 mRNA levels were not up-regulated as shown in FIG. 3B.

C. miRNA-196 Represses HCV Non-Structural NS5A Protein Expression in 9-13 Cells Expressing HCV Non-Structural Proteins

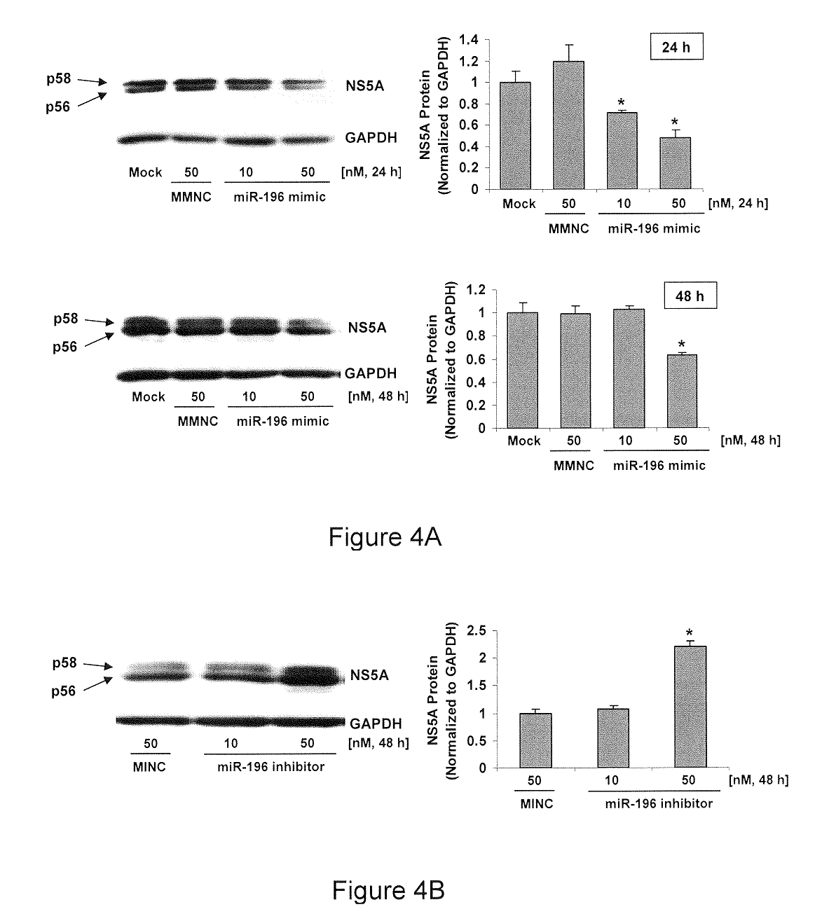

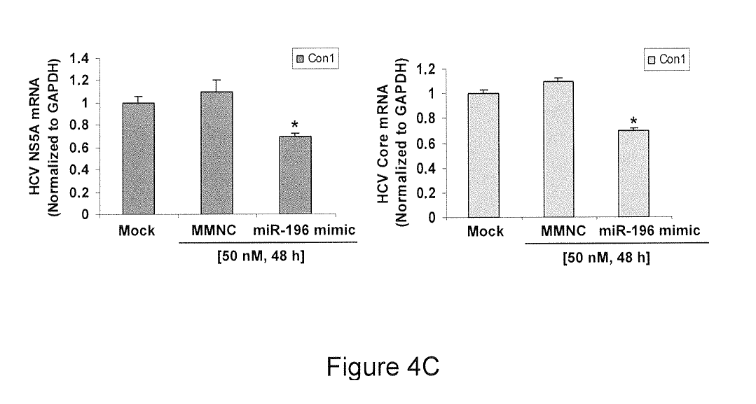

Until the present invention, it was not known if miRNA-196 alters HCV non-structural NS5A protein levels in human hepatoma cells expressing HCV non-structural proteins. To determine whether miRNA-196 alters HCV non-structural NS5A protein levels in human hepatoma cells expressing HCV non-structural proteins, 9-13 cells were transfected with either miRNA-196 mimic or inhibitor. At 24 h and 48 h after transfection, Western blots were performed to analyze NS5A and GAPDH protein levels. 50 nM miRNA-196 mimic resulted in a significant reduction in the expression of NS5A protein levels by about 52% after 24 h transfection, and about 37% after 48 h, compared with the same amount of miRNA mimic negative control as shown in FIG. 4A. In contrast, down-regulation of miRNA-196 by miRNA-196 specific inhibitor significantly increased NS5A protein levels by 2.2 fold as shown in FIG. 4B. To evaluate whether miRNA-196 may inhibit HCV replication, Con1 full-length HCV replicon Huh-7.5 cells were transfected with negative controls or miRNA-196 mimic for 48 hours. As shown in FIG. 4C, miRNA-196 resulted in a significant reduction in viral RNA levels, in comparison with miRNA mimic negative control (MMNC).

D. miR-196 Inhibits HCV Expression in the HCV JFH1-Based Cell Culture System

Huh-7.5 cells were transfected with 2 μg/well of HCV J6/JFH1 RNA by Lipofectamine 2000. After 48 h, cells were transfected with miRNA-196 mimic, or miRNA mimic negative control (MMNC). For HCV infection, naïve Huh-7.5 were cultured with 1 mL of cell culture supernatants harvested from J6/JFH1-transfected cells, as previously described. After 48 hours of exposure to the supernatants, cells were transfected with miRNA-196 mimic, or miRNA mimic negative control (MMNC) for 48 h. Cells were harvested and total RNA and proteins were extracted. HCV RNA was quantified by qRT-PCR, and HCV NS3 and GAPDH protein levels were measured by Western blots.

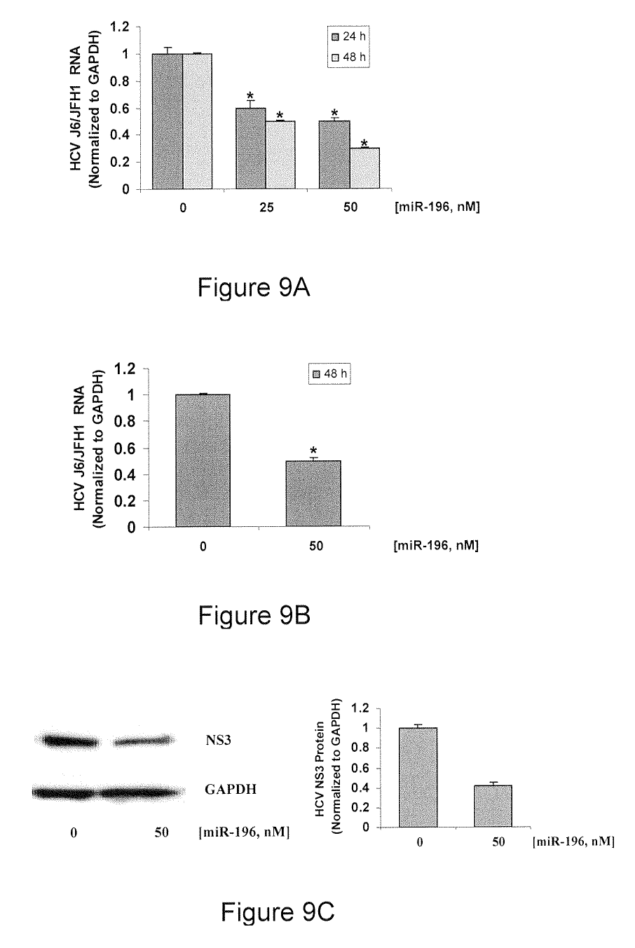

A perfect match for miRNA-196 was found in the coding region of the HCV NS5A gene in HCV JFH1 genome. A down-regulatory effect of miRNA-196 on HCV expression in the HCV J6/JFH1 cell culture system was observed. 50 nM of miRNA-196 led to a significant decrease of HCV J6/JFH1 RNA by nearly 70% in J6/JFH1 transfected Huh-7.5 cells (as shown in FIG. 9A), ˜50% in J6/JFH1 infected Huh-7.5 cells (as shown in FIG. 9B), and ˜60% reduction of HCV NS3 protein in J6/JFH1 infected Huh-7.5 cells (as shown FIG. 9C). These results were consistent with previous observations in the JFH1 cell culture system.

E. miRNA-196 Directly Interacts with the 3′-UTR of Bach1 mRNA in 9-13 Cells Expressing HCV Non-Structural Proteins

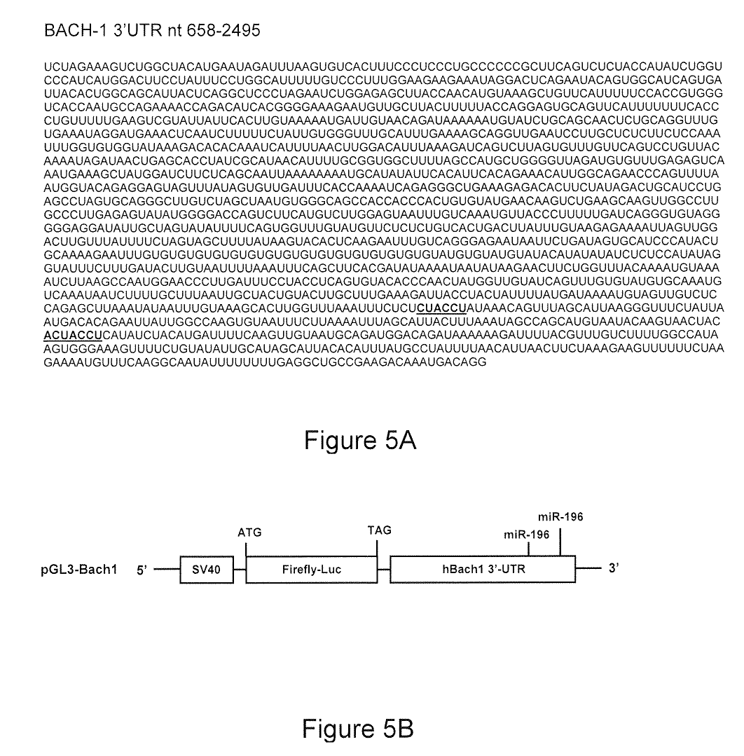

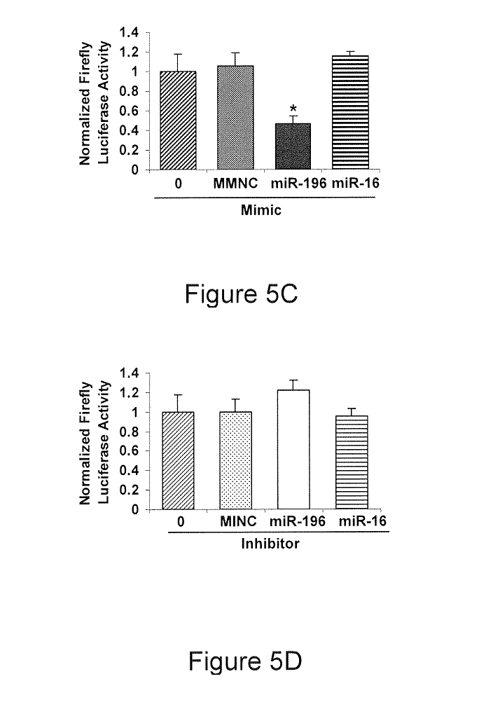

To further establish that miRNA-196 targets the 3′-UTR of Bach1 mRNA, which contains two predicted seed match sites for miR-196 as shown in FIG. 5A. Rather than exerting a less direct and specific regulation, a reporter construct called pGL3-Bach1 with Bach1 3′-UTR downstream of the firefly luciferase (f-luc) open reading frame (FIG. 5B) was used. 9-13 cells were co-transfected with pGL3-Bach1 (f-luc), pRL-TK (renilla, to normalize for transfection efficiencies), and with miRNA-negative controls, miRNA-196 mimic or inhibitor or miRNA-196 (a “negative” miR with no predicted binding sites in the 3′-UTR of Bach1 mRNA). 48 h after transfection, the luciferase reporter activity was assayed. miRNA-196 mimic transfection significantly decreased reporter activity by about 53%, whereas miRNA mimic negative control and miRNA-196 mimic had no effect on reporter luciferase activity. Additionally, no significant change of reporter activity was observed in cells transfected with miRNA-196 inhibitor. As shown in FIG. 5C, miRNA-196 mimic inhibited the f-luc activities of pGL3-Bach1 reporter, while miRNA-196 inhibitor slightly increased the f-luc activity of pGL3-Bach1 reporter (FIG. 5D).

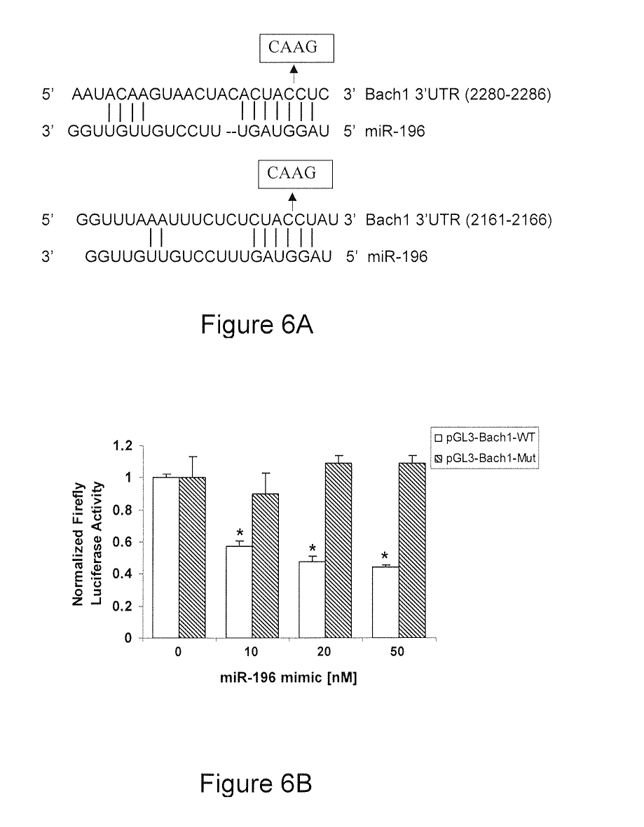

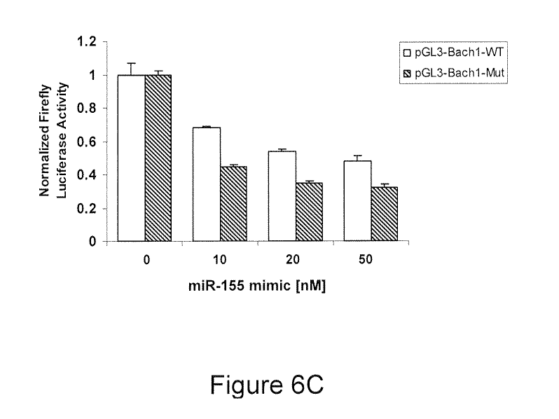

Next, 9-13 cells were co-transfected with Luc reporter construct containing a four nucleotide mutant Bach1 3′-UTR, which was called pGL3-Bach1-Mut (FIG. 6A), and with pRL-TK, and with miRNA-196 mimic or miRNA-155 mimic (a “negative” miR, with no changes of predicted miR-155 binding sites in pGL3-Bach1-WT and pGL3-Bach1-Mut). 48 hours after transfection, the luciferase reporter activities were measured using Dual Luciferase Assay System from Promega, firefly luciferase activities were normalized to renilla luciferase activities and total protein. As shown in FIG. 6B, miRNA-196 mimic decreased the f-luc activity of pGL3-Bach1-WT but not pGL-Bach1-Mut reporter. miRNa-196 mimic transfection significantly decreased luciferase activity in a dose-dependent fashion, whereas miRNA-196 did not change reporter activity in cells transfected with the reporter construct containing mutant binding sites for miRNA-196. miRNA-155 significantly decreased reporter activity in both pGL3-Bach1-WT and pGL3-Bach1-Mut as shown in FIG. 6C. These results demonstrate that miRNA-196 directly regulates Bach1 gene expression and miRNA-196 mediates down-regulation of Bach1 though the 3′-UTR of Bach1 mRNA.

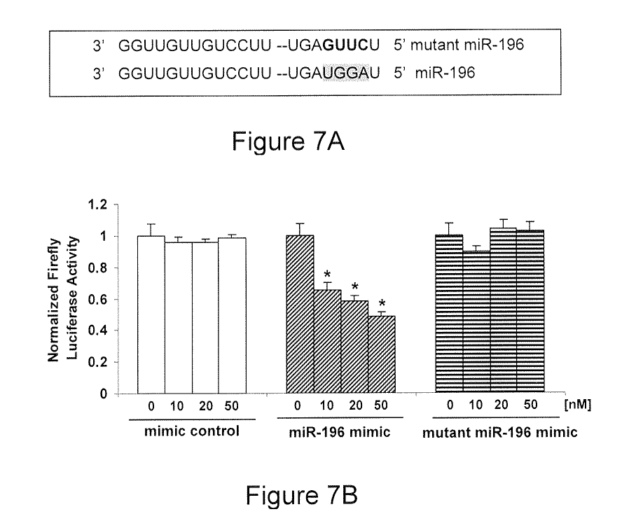

To further determine the direct interaction between miRNA-196 and Bach1-3′-UTR, four nucleotide mutants were introduced to produce mutant miRNA 196 as shown in FIG. 7A, in which seed match sites for the 3′-UTR of Bach1 mRNA were abolished. Cells were co-transfected with pGL3-Bach1-WT, and with pRL-TK, and with increasing concentrations of miRNA mimic negative control, miRNA-196 mimic or mutant miRNA-196 mimic, the luciferase reporter activity was assayed. As shown in FIG. 7B, miRNA-196 resulted in a significant reduction in luciferase activity in a dose-dependent fashion, which was constant with our previous observations, whereas miRNA negative controls and mutant miR-196 did not affect luciferase activity, further indicating the direct interaction between miR-196 and the 3′-UTR of Bach1 mRNA.

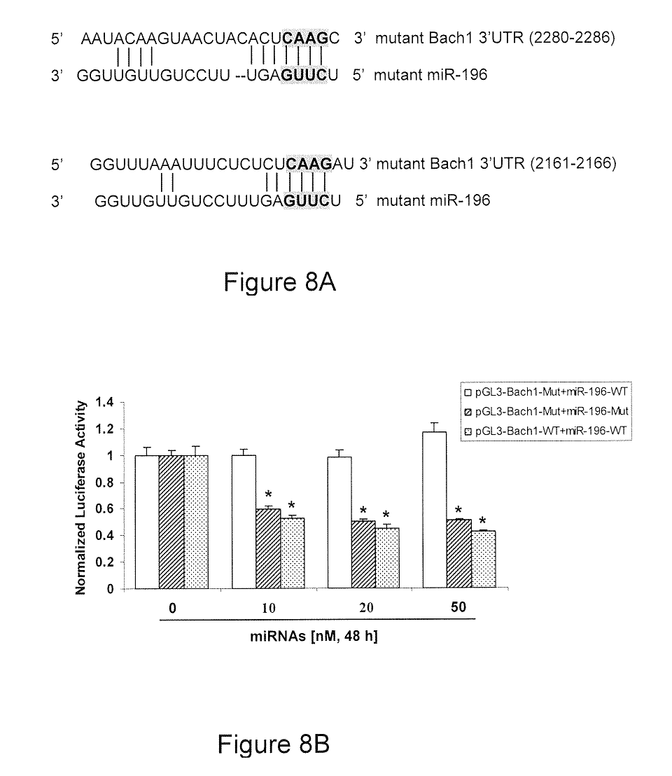

Mutant miRNA-196 (miR-196-Mut), containing base complementary with mutant pGL3-Bach1 (pGL3-Bach1-Mut), should restore its effect on mutant reporter (Bach1-3′-UTR-Mut) activity since they again match perfectly in their seed regions as shown in FIG. 8A. Mutant miRNA-196 mimic significantly inhibited luciferase activity in cells transfected with mutant pGL3-Bach1, which was mutated to “fit” mutant miRNA-196. On the other hand, no significant effects of miRNA-196 wild type on mutant reporter (pGL3-Bach1-Mut) luciferase activity were observed. Thus, further proving the direct interaction between miRNA-196 and the 3′-UTR of Bach1 mRNA as illustrated by FIG. 8B.

The experimental work described above illustrates that the transfection of miRNA-196 mimic significantly down-regulated Bach1 and HCV non-structural NS5A protein levels, and up-regulated HMOX1 gene expression. As such, miRNA-196 can play an important, perhaps even critical, role in the regulation of HCV replication and HMOX1/Bach1 expression in hepatocytes. Accordingly, up-regulation of miR-196 can provide an additional new therapeutic approach for therapy of chronic hepatitis C and, perhaps, other hepatic disorders.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A method of treating a mammal suffering from HCV infection, comprising reducing the expression of Bach1 protein levels in infected cells expressing HCV.

2. The method of claim 1, wherein reducing the expression of Bach1 protein levels in cells expressing HCV non-structural proteins comprises transfecting the cells with miRNA-196 mimic so that miRNA-196 binds with the 3′-UTR of Bach1 mRNA to reduce the expression of Bach1.

3. The method of claim 2, further comprising up-regulating or over-expressing miRNA-196 or miRNA-196 mimic.

4. The method of claim 2, wherein miRNA-196 is up-regulated by administering interferon beta.

5. The method of claim 2, wherein the expression of Bach1 protein levels is reduced by about 10% to about 75%.

6. The method of claim 2, wherein the expression of Bach1 protein levels 24 hours after transfection comprises a reduction of about 50% to about 60%.

7. The method of claim 2, wherein the expression of Bach1 protein levels 48 hours after transfection comprises a reduction of about 60% to about 70%.

8. A method of treating a mammal suffering from HCV infection, comprising up-regulating HMOX1 gene expression in cells infected with HCV.

9. The method of claim 8, further comprising down-regulating Bach1 gene expression in the cells.

10. The method according to claim 9, wherein Bach1 gene expression is down-regulated by transfecting the cells with miRNA-196 mimic.

11. The method of claim 10, further comprising up-regulating or over-expressing miRNA-196.

12. The method of claim 8, wherein the cells comprise hepatocytes.

13. The method of claim 11, wherein the expression of HMOX1 is increased by about 2 to about 3 fold over the level of HMOX1 expression in cells that have not been transfected with miRNA-196 mimic.

14. A method of treating a mammal suffering from HCV infection, comprising reducing the expression of HCV RNA and protein expression in HCV replicon cells and HCV infected cells by transfecting the cells with miRNA-mimic.

15. The method of claim 14, further comprising up-regulating or over-expressing miRNA-196.

16. The method of claim 15, wherein the expression of HCV NS5A protein levels is reduced by 10 to 60 percent.

17. The method of claim 15, wherein the expression of HCV NS5A protein levels 24 hours after transfection comprises a reduction of about 45 to about 55 percent.

18. The method of claim 15, wherein the expression of HCV NS5A protein levels 48 hours after transfection comprises a reduction of about 35 to about 45 percent.

19. A formulation for the treatment of cells expressing HCV non-structural proteins and mammals having hepatitis C, comprising a therapeutically effective amount of miRNA-196 mimic such that Bach1 and HCV NS5A expression levels are down-regulated while HMOX1-expression is increased; wherein the formulation is adapted to enable the transfection of miRNA-196 mimic into hepatocytes expressing HCV proteins.

20. The formulation of claim 19, wherein the miRNA-196 mimic is included in liposomes so that the miRNA-196 mimic can be released into cells expressing HCV proteins.

21. The formulation of claim 20, wherein the formulation comprises cationic lipids.

22. The formulation of claim 19, further comprising cationic amphiphiles so that the miRNA-196 mimic can be released into cells expressing HCV proteins.


SEQUENCE LISTING (29 sequences)

SEQ ID NO: 1 - RNA, 23 nt, Homo sapiens
  Bach1 3′UTR (2280-2286 bp)
aauacaaguaacuacacuaccuc

SEQ ID NO: 2 - RNA, 21 nt, Homo sapiens
  miR-196
gguuguuguccuuugauggau

SEQ ID NO: 3 - RNA, 34 nt, Homo sapiens
agcauguaauacaaguaacuacacuaccucauau

SEQ ID NO: 4 - RNA, 38 nt, Mus musculus
agcauguaauacaaguaacuacacuaccucauacacac

SEQ ID NO: 5 - RNA, 38 nt, Rattus rattus
agcauguaauacaagugacuacacuaccucauccguac

SEQ ID NO: 6 - RNA, 34 nt, Gallus gallus
agcauguaauacaaguaacuacacuaccucauac

SEQ ID NO: 7 - RNA, 32 nt, Canis familiaris
agcaccuaauacaagugacuacacuaccucac

SEQ ID NO: 8 - RNA, 23 nt, Homo sapiens
  Bach1 3′UTR (2161-2166)
gguuuaaauuucucucuaccuau

SEQ ID NO: 9 - RNA, 21 nt, Homo sapiens
  miR-196
gguuguuguccuuugauggau

SEQ ID NO: 10 - RNA, 37 nt, Homo sapiens
aaagcacuugguuuaaauuucucucuaccuauaaaca

SEQ ID NO: 11 - RNA, 33 nt, Mus musculus
aagacacuugguuuaaauuucucucuaugaaca

SEQ ID NO: 12 - RNA, 37 nt, Rattus rattus
uagacacuugguuuaaauuucucucuauucaugaaca

SEQ ID NO: 13 - RNA, 37 nt, Gallus gallus
aagacacuugguuuaaauuucucucuacauauaaaca

SEQ ID NO: 14 - RNA, 37 nt, Canis familiaris
aagaaacuucuuggcaauuuuuucuucuuacuaggua

SEQ ID NO: 15 - RNA, 1836 nt, Homo sapiens
  BACH-1 3′UTR (nt 658-2495)
ucuagaaagucuggcuacaugaauagauuuaagugucacuuucccucccugccccccgcuucagucucuaccauaucuggucccaucauggacuuccuauuuccuggcauuuuugucccuuuggaagaagaaauaggacucagaauacaguggcaucagugauuacacuggcagcauuacucaggcucccuagaaucuggagagcuuaccaacauguaaagcuguucauuuuuccaccgugggucaccaaugccagaaaaccagacaucacggggaaagaauguugcuuacuuuuuaccaggagugcaguucauuuuuuucacccuguuuuugaagucguauuauucacuuguaaaaaugauuguaacagauaaaaaauguaucugcagcaacucugcagguuugugaaauaggaugaaacucaaucuuuuucuauuguggguuugcauuugaaaagcagguugaauccuugcucucuucuccaaauuuggugugguauaaagacacacaaaucauuuuaacuuggacauuuaaagaucagucuuaguguuuguucaguccuguuacaaaauagauaacugagcaccuaucgcauaacauuuugcgguggcuuuuagccaugcugggguuagauguguuugagagucaaaugaaagcuauggaucuucucagcaauuaaaaaaaaugcauauauucacauucacagaaacauuggcagaacccaguuuuaaugguacagaggaguaguuuauaguguugauuucaccaaaaucagagggcugaaagagacacuucuauagacugcauccugagccuagugcagggcuugucuagcuaaugugggcagccaccacccacuguguaugaacaagucugaagcaaguuggccuugcccuugagaguauauggggaccagucuucaugucuuggaguaauuugucaaauguuacccuuuuugaucaggguguagggggaggauauugcuaguauauuuucagugguuuguauguucucucugucacugacuuauuuguaagagaaaauuaguuggacuuguuuauuuucuaguagcuuuuauaaguacacucaagaauuugucagggagaauaauucugauagugcaucccauacugcaaaagaauuuguguguguguguguguguguguguguguguguguauguguauguauacauauauaucucuccauauagguauuucuuugauacuuguaauuuuaaauuucagcuucacgauauaaaauaauauaagaacuucugguuuacaaaauguaaaaucuuaagccaauggaacccuugauuuccuaccucaguguacacccaacuaugguuguaucaguuuguguaugugcaaaugucaaauaaucuuuugcuuuaauugcuacuguacuugcuuugaaagauuaccuacuauuuuaugauaaaauguaguugucuccagagcuuaaauauaauuuguaaagcacuugguuuaaauuucucucuaccuauaaacaguuuagcauuaaggguuucuauuaaugacacagaauuauuggccaaguguaauuucuuaaaauuuagcauuacuuuaaauagccagcauguaauacaaguaacuacacuaccucauaucuacaugauuuucaaguuguaaugcagauggacagauaaaaaagauuuuacguuugucuuuuggccauaagugggaaaguuuucuguauauugcauagcauuacacauuuaugccuauuuuaacauuaacuucuaaagaaguuuuuucuaagaaaauguuucaaggcaauauuuuuuuugaggcugccgaagacaaaugacagg

SEQ ID NO: 16 - DNA, 4 bp, Artificial Sequence
  Four replacement nucleotides in the two seed match sites of BACH-1 3′UTR
caag

SEQ ID NO: 17 - RNA, 21 nt, Artificial Sequence
  5′ mutant miR-196
gguuguuguccuuugaguucu

SEQ ID NO: 18 - RNA, 21 nt, Homo sapiens
  5′ miR-196
gguuguuguccuuugauggau

SEQ ID NO: 19 - RNA, 23 nt, Artificial Sequence
  3′ mutant Bach1 3′UTR (2280-2286)
aauacaaguaacuacacucaagc

SEQ ID NO: 20 - RNA, 21 nt, Artificial Sequence
  5′ mutant miR-196
gguuguuguccuuugaguucu

SEQ ID NO: 21 - RNA, 23 nt, Artificial Sequence
  3′ mutant Bach1 3′UTR (2161-2166)
gguuuaaauuucucucucaagau

SEQ ID NO: 22 - DNA, 22 bp, Artificial Sequence
  Bach-1-specific sense primer
ggacactccttgccaaatgcag

SEQ ID NO: 23 - DNA, 22 bp, Artificial Sequence
  Bach-1-specific anti-sense primer
tgacctggttctgggctctcac

SEQ ID NO: 24 - DNA, 18 bp, Artificial Sequence
  HMOX1-specific sense primer
cgggccagcaacaaagtg

SEQ ID NO: 25 - DNA, 22 bp, Artificial Sequence
  HMOX1-specific anti-sense primer
agtgtaaggacccatcggagaa

SEQ ID NO: 26 - DNA, 17 bp, Artificial Sequence
  Cul3-specific sense primer
gtgctcacgacaggata

SEQ ID NO: 27 - DNA, 21 bp, Artificial Sequence
  Cul3-specific anti-sense primer
gtttggctaagtagaaccttc

SEQ ID NO: 28 - DNA, 19 bp, Artificial Sequence
  GAPDH-specific sense primer
ttgttgccatcaatgaccc

SEQ ID NO: 29 - DNA, 19 bp, Artificial Sequence
  GAPDH-specific anti sense primer
cttcccgttctcagccttg